(19) United States
(12) Patent Application Publication
(10) Pub. No.: US 2019/0015354 A1
(43) Pub. Date: Jan. 17, 2019

(54) INOSITOL-CONTAINING COMESTIBLE UNITS AND METHODS OF TREATMENT USING THE SAME

(71) Applicant: Richard Louis Price, Suffern, NY (US)

(72) Inventor: Richard Louis Price, Suffern, NY (US)

(21) Appl. No.: 16/132,742

(22) Filed: Sep. 17, 2018

Related U.S. Application Data
(63) Continuation-in-part of application No. 15/346,079, filed on Nov. 8, 2016, now Pat. No. 10,098,848.
Application No. 15/346,079 is a continuation-in-part of application No. 14/596,726, filed on Jan. 14, 2015, now abandoned.
Application No. 14/596,726 is a continuation-in-part of application No. 14/245,121, filed on Apr. 4, 2014, now Pat. No. 9,603,812.
Application No. 14/245,121 is a continuation-in-part of application No. 14/166,483, filed on Jan. 28, 2014, now abandoned.
Application No. 14/166,483 is a continuation-in-part of application No. 13/860,824, filed on Apr. 11, 2013, now Pat. No. 9,211,284.

(51) Int. Cl. A61K 31/047; A61P 25/00; A21D 2/18; A21D 13/80; A23L 33/125

(52) U.S. Cl. CPC A61K 31/047; A61P 25/00; A23V 2002/00; A21D 13/80; A23L 33/125; A21D 2/181

(57) ABSTRACT

A method for treating a patient having a psychiatric disorder and/or certain psychological or neurological symptoms is disclosed. The method includes administering to the patient one or more comestible units, e.g., cookies, cumulatively comprising a therapeutically effective amount of inositol, optionally at least 12,000 mg inositol.

### CROSS-REFERENCE TO RELATED APPLICATIONS

This application is a continuation-in-part of U.S. patent application Ser. No. 15/346,079, entitled “Inositol-Containing Comestible Units and Methods of Treatment Using the Same,” filed Nov. 8, 2016 and currently pending, which is a continuation-in-part of U.S. patent application Ser. No. 14/596,726, entitled “Inositol-Containing Comestible Units and Methods of Treating Autistic Spectrum Disorder Using the Same,” filed Jan. 14, 2015, which is a continuation-in-part of U.S. patent application Ser. No. 14/245,121, entitled “Treatment of Autistic Spectrum Disorder, filed Apr. 4, 2014 (now U.S. Pat. No. 9,603,812), which is a continuation-in-part of U.S. patent application Ser. No. 14/166,483, entitled “Diagnosis and Treatment of a Form of Autistic Spectrum Disorder, filed Jan. 28, 2014, which is a continuation-in-part of U.S. patent application Ser. No. 13/860,824, entitled “Diagnosis and Treatment of P.R.I.C.E. Syndrome,” filed Apr. 11, 2013 (now U.S. Pat. No. 9,211,284), all of which are incorporated by reference herein in their entireties.

### BACKGROUND OF THE INVENTION

### 1. Field of Invention

The invention relates to treatment of psychiatric or neurological symptoms that may or may not relate to an underlying psychiatric disorder recognized in the DSM 5.0, e.g., Autistic Spectrum Disorder, Anxiety, Obsessive-Compulsive Disorder. More particularly, the invention relates to administering to a person having one or more symptoms (whether or not related to an underlying psychiatric and/or neurological condition) that respond to therapeutic doses of inositol provided in one or more comestible units, e.g., cookies. Such symptoms may include, among others, anxiety, hypersensitivity, restricted areas of interest, repetitive behaviors, irritability and emotional lability.

### 2. Description of Related Art

When a psychiatrist is presented with a patient exhibiting one or more behaviors such as poor social skills, defiance, lack of patience, difficulty paying attention, ritualistic behavior and/or mood swings, where such behavior(s) interferes with normal functioning, the psychiatrist must first make a diagnosis before formulating a treatment plan.

Today, the psychiatrist's nomenclature, i.e., the criteria for psychiatric evaluation and classification is provided in the Diagnostic and Statistical Manual of Mental Disorders (“DSM”), a periodically revised psychiatric “Bible” published by the American Psychiatric Association. The current version of the DSM is DSM 5.0, which was published on May 18, 2013.

The psychiatrist's professional judgment in rendering a diagnosis is largely informed by the criteria for various disorders set forth in the DSM. Thus, a psychiatrist presented with a patient exhibiting any such symptoms as those described above would consult the DSM in rendering a diagnosis. The diagnosis would, in turn, inform a treatment program. Whether a given patient is determined, for example, to have ADHD as opposed to Hypomania in Bipolar Disorder, depends on whether the patient's symptoms comport with criteria set forth for these conditions in the DSM. Proper diagnosis is critical since a wrong diagnosis will likely lead to an ineffective or even potentially harmful treatment program.

The DSM 5.0 definition of Autism Spectrum Disorder (“ASD”) is as follows:
A. Persistent deficits in social communication and social interaction across contexts, not accounted for by general developmental delays, and manifest by all 3 of the following:
   1. Deficits in social-emotional reciprocity: ranging from abnormal social approach and failure of normal back and forth conversation through reduced sharing of interests, emotions, and affects and response to total lack of initiation of social interaction.
   2. Deficits in nonverbal communicative behaviors used for social interaction: ranging from poorly integrated verbal and nonverbal communication, through abnormalities in eye contact and body language, or deficits in understanding and use of nonverbal communication, to total lack of facial expression or gestures.
   3. Deficits in developing and maintaining relationships appropriate to developmental level (beyond those of caregivers); ranging from difficulties to adjusting behavior to suit different social contexts through difficulties in sharing imaginative play and in making friends to an apparent absence of interest in people.
B. Restricted, repetitive patterns of behavior, interests, or activities as manifested by at least two of the following:
   1. Stereotyped or repetitive speech, motor movements, or use of objects (such as simple motor stereotypies, echolalia, repetitive use of objects, or idiosyncratic phrases).
   2. Excessive adherence to routines, ritualized patterns of verbal or nonverbal behavior, or excessive resistance to change (such as motoric rituals, insistence on same route or food, repetitive questioning or extreme distress at small changes).
   3. Highly restricted, fixated interests that are abnormal in intensity or focus (such as strong attachment to or preoccupation with unusual objects, excessively circumscribed or perseverative interests).
   4. Hyper- or hypo-reactivity to sensory input or unusual interest in sensory aspects of environment (such as apparent indifference to pain/heat/cold, adverse response to specific sounds or textures, excessive smelling or touching of objects, fascination with lights or spinning objects).
C. Symptoms must be present in early childhood (but may not become fully manifest until social demands exceed limited capacities).
D. Symptoms together limit and impair everyday functioning.

Part A of the DSM 5.0 definition of ASD and Part B of the DSM 5.0 definition of ASD are hereinafter collectively referred to as “core symptoms” of ASD or “core ASD symptoms,” since they are, by definition, present in all ASD patients. Throughout this specification, the constituent symptoms of the core symptoms of ASD may be individually referred to respectively as “part A of the DSM 5.0 definition of ASD” and “part B of the DSM 5.0 definition of ASD.”

Other symptoms that may be manifest in ASD patients and which are associated with their ASD are referred to herein as “associated symptoms” of ASD or “associated ASD symptoms”. Such associated symptoms of ASD may include at least one of the following: impulsivity, concentration deficit or attention deficit and emotional lability/irritability. As discussed below, as relates to an optional aspect of the invention, some of these symptoms may also manifest themselves in patients with a different underlying condition or with no particular diagnosed underlying condition.

The term “impulsivity,” as used herein, is characterized by the following: often blurts out answers before questions have been completed and/or often has difficulty waiting for his/her turn and/or often interrupts or intrudes on others (e.g., butts into conversations or games).

The terms “concentration deficit” and “attention deficit” are synonymous with each other and are therefore interchangeable. As used herein, the terms “concentration deficit” and “attention deficit” are characterized by the following: deficits in concentration as evidenced by often having difficulty sustaining attention in tasks or play activities, often does not seem to listen when spoken to directly, is often easily distracted by extraneous stimuli.

The terms “emotional lability” and “irritability,” are synonymous with each other and are therefore interchangeable. As used herein, “emotional lability” and “irritability” are sometimes compounded into the single term (which is synonymous with each individual term): “emotional lability/irritability.” Emotional lability/irritability is characterized by the following: severe, reactive mood swings in response to real or perceived situations where demanded needs are not being met in the environment. Emotional lability/irritability may optionally be measured using the Aberrant Behavior Checklist irritability subscale.

The Applicant has discovered a pervasive subpopulation within ASD that responds particularly well to a combination therapy that the Applicant has invented. In addition to the core symptoms of DSM 5.0 ASD, defined above, the Applicant has found patients in this subpopulation have one or more (usually all) associated symptoms of ASD, as defined above (i.e., impulsivity, concentration deficit or attention deficit and/or emotional lability/irritability). The combination therapy to treat ASD that Applicant invented, namely administering a therapeutically effective amount of an alpha-2 adrenergic agonist in an extended release dosage form in combination with a therapeutically effective amount of inositol, is described in detail in the following of Applicant's patent applications, all of which are incorporated by reference herein in their entireties: WO 2014/168820 and U.S. Pat. Pub. Nos. 2017/0348417, 2014/0309271, 2014/0309270 and 2014/0309269.

Based on Applicant's experience and insights, the aforementioned combination therapy is a superior treatment for ASD compared to use of either agent alone. For example, neither agent alone is effective in treating all core symptoms of ASD and associated symptoms of ASD, whereas the combination is effective in treating all such symptoms. Moreover, Applicant has found that the combination exhibits a synergistic effect in treating Part A of the DSM 5.0 definition of ASD and emotional lability/irritability.

Notwithstanding the superior efficacy of the combination over administration of either agent alone in ASD, monotherapy with only one of these agents is still far more effective than either doing nothing or administering other known drugs. Applicant has seen cases in which ASD patients were misdiagnosed based on patients' presenting associated symptoms of ASD, which treating physicians misinterpreted as being indicative of underlying conditions unrelated to ASD. Such misdiagnoses led to prescribing drugs that either did not help, or exacerbated these patients' symptoms, and/or caused serious side effects. Such drugs included psychostimulants, Strattera, Wellbutrin, Provigil, Nuvigil, Tenex, propranonol, selective serotonin re-uptake inhibitors, serotonin and norepinephrine reuptake inhibitors, mood stabilizers and atypical antipsychotics. See, e.g., Examples 1 and 2 of Applicant's U.S. Pat. Pub. 2014/0309270.

Whereas the aforementioned drugs were found to be ineffective or harmful to ASD patients, Applicant has found that an extended release alpha-2 adrenergic agonist, such as extended release clonidine (e.g., marketed as KAPVAY®) or extended release guanfacine (e.g., marketed as INTUNIV®) as monotherapy for ASD is effective in treating some ASD symptoms. Likewise, Applicant has found that high doses of inositol are effective as a monotherapy in treating some ASD symptoms. While Applicant's combination therapy is superior and preferred for treating ASD over monotherapy using either agent alone, such monotherapy is still an improvement over anything else known to Applicant (aside of course from Applicant's combination therapy). Given the urgency of the health need for effective ASD treatment, and the fact that regulatory approval of any new drug (in this case, the combination therapy) generally takes years, there is a need for a readily available treatment for ASD patients.

### Attention Deficit Hyperactivity Disorder

The DSM 5.0 definition of Attention Deficit Hyperactivity Disorder (“ADHD”) is as follows:
A. A persistent pattern of inattention and/or hyperactivity-impulsivity that interferes with functioning or development, as characterized by (1) and/or (2):
   1. Inattention: Six (or more) of the following symptoms have persisted for at least 6 months to a degree that is inconsistent with developmental level and that negatively impacts directly on social and academic/occupational activities:
      Note: The symptoms are not solely a manifestation of oppositional behavior, defiance, hostility, or failure to understand tasks or instructions. For older adolescents and adults (age 17 and older), at least five symptoms are required.
   a. Often fails to give close attention to details or makes careless mistakes in schoolwork, at work, or during other activities (e.g., overlooks or misses details, work is inaccurate).
   b. Often has difficulty sustaining attention in tasks or play activities (e.g., has difficulty remaining focused during lectures, conversations, or lengthy reading).
   c. Often does not seem to listen when spoken to directly (e.g., mind seems elsewhere, even in the absence of any obvious distraction).
   d. Often does not follow through on instructions and fails to finish schoolwork, chores, or duties in the workplace (e.g., starts tasks but quickly loses focus and is easily sidetracked).
   e. Often has difficulty organizing tasks and activities (e.g., difficulty managing sequential tasks; difficulty keeping materials and belongings in order; messy, disorganized work; has poor time management; fails to meet deadlines).
   f. Often avoids, dislikes, or is reluctant to engage in tasks that require sustained mental effort (e.g., schoolwork or homework; for older adolescents and adults, preparing reports, completing forms, reviewing lengthy papers).
   g. Often loses things necessary for tasks or activities (e.g., school materials, pencils, books, tools, wallets, keys, paperwork, eyeglasses, mobile telephones).
   h. Is often easily distracted by extraneous stimuli (for older adolescents and adults, may include unrelated thoughts).
   i. Is often forgetful in daily activities (e.g., doing chores, running errands; for older adolescents and adults, returning calls, paying bills, keeping appointments).
   2. Hyperactivity and impulsivity: Six (or more) of the following symptoms have persisted for at least 6 months to a degree that is inconsistent with developmental level and that negatively impacts directly on social and academic/occupational activities:
      Note: The symptoms are not solely a manifestation of oppositional behavior, defiance, hostility, or a failure to understand tasks or instructions. For older adolescents and adults (age 17 and older), at least five symptoms are required.
   a. Often fidgets with or taps hands or feet or squirms in seat.
   b. Often leaves seat in situations when remaining seated is expected (e.g., leaves his or her place in the classroom, in the office or other workplace, or in other situations that require remaining in place).
   c. Often runs about or climbs in situations where it is inappropriate. (Note: In adolescents or adults, may be limited to feeling restless.)
   d. Often unable to play or engage in leisure activities quietly.
   e. Is often “on the go,” acting as if “driven by a motor” (e.g., is unable to be or uncomfortable being still for extended time, as in restaurants, meetings; may be experienced by others as being restless or difficult to keep up with).
   f. Often talks excessively.
   g. Often blurts out an answer before a question has been completed (e.g., completes people's sentences; cannot wait for turn in conversation).
   h. Often has difficulty waiting his or her turn (e.g., while waiting in line).
   i. Often interrupts or intrudes on others (e.g., butts into conversations, games, or activities; may start using other people's things without asking or receiving permission; for adolescents and adults, may intrude into or take over what others are doing).
B. Several inattentive or hyperactive-impulsive symptoms were present prior to age 12 years.
C. Several inattentive or hyperactive-impulsive symptoms are present in two or more settings (e.g., at home, school, or work; with friends or relatives; in other activities).
D. There is clear evidence that the symptoms interfere with, or reduce the quality of, social, academic, or occupational functioning.
E. The symptoms do not occur exclusively during the course of schizophrenia or another psychotic disorder and are not better explained by another mental disorder (e.g., mood disorder, anxiety disorder, dissociative disorder, personality disorder, substance intoxication or withdrawal).

Part A(1) of the DSM 5.0 definition of ADHD and Part A(2) of the DSM 5.0 definition of ADHD are hereinafter collectively referred to as “core symptoms” of ADHD or “core ADHD symptoms,” since either one or both of them are, by definition, present in all ADHD patients. Throughout this specification, the constituent symptoms of the core symptoms of ADHD may be individually referred to respectively as: (1) “part A(1) of the DSM 5.0 definition of ADHD,” alternatively referred to as “Inattention in ADHD”; and (2) “part B of the DSM 5.0 definition of ADHD,” alternatively referred to as “Hyperactivity and Impulsivity in ADHD”.

Other symptoms that may be manifest in ADHD patients and which are associated with their ADHD are referred to herein as “associated symptoms” of ADHD or “associated ADHD symptoms”. Such associated symptoms of ADHD may include at least one of the following: social reciprocity deficits, emotional lability/irritability, insomnia, constipation, and hyperfocus. These are now explained.

The term “social reciprocity deficits associated with ADHD” as used herein carries a similar meaning to part A of DSM 5.0 ASD, except for the underlying disorder with which such symptomology is associated. That is, the severity of the social reciprocity deficits symptomology is sub-threshold for an ASD diagnosis and/or does not meet full clinical criteria for ASD.

The term “emotional lability/irritability associated with ADHD” as used herein carries a similar meaning as provided above for “emotional lability/irritability” as an associated ASD symptom, except for the underlying disorder with which such symptomology is associated. The term “emotional lability/irritability associated with ADHD” is also synonymous with “impulsive aggression,” which was a term coined in the ADHD literature in recent years.

The term “insomnia associated with ADHD” carries the traditional medical definition of insomnia, except that it is associated with the patient's ADHD.

The term “constipation associated with ADHD” carries the traditional medical definition of constipation, except that it is associated with the patient's ADHD.

The term “hyperfocus associated with ADHD” carries the traditional medical definition describing the phenomenon of some ADHD patients who hyperfocus as a result of their underlying condition and/or ADHD patients who manifest highly restricted, fixated interests that are abnormal in intensity or focus as a result of taking an extended release alpha-2-adrenergic agonist for treatment of the patient's ADHD.

Inositol is a natural supplement that is already widely available, e.g., in vitamin stores, without a prescription. Applicant has discovered that high doses of inositol are safe and effective in treating Part B of the DSM 5.0 definition of ASD and the associated symptom of ASD of emotional lability/irritability. Applicant has also discovered that high doses of inositol directly addresses and one or more associated symptoms of ADHD, including one or more of: social reciprocity deficits, emotional lability/irritability, insomnia, constipation and hyperfocus. Stimulant medications can help core ADHD symptoms, but unlike inositol, can exacerbate hyperfocus, emotional lability/irritability, restricted areas of interest/repetitive behavior and hypersensitivity. Alleviation of these symptoms alone would provide some relief to ASD and ADHD patients and their caretakers. While there are exceptions, Applicant has found that therapeutic doses of inositol for ASD patients tend to be about 9,000 mg to about 32,400 mg per day, more typically about 18,000 mg to about 32,400 mg per day. Inositol is typically available as a supplement, e.g., in powder form. Given the large amounts of inositol generally necessary to provide therapeutic doses to ASD patients, such commercially available inositol is not typically administered orally without being mixed with some other substance. For example, inositol is typically mixed into a drink or semi solid food, e.g., applesauce.

However, Applicant has found that children sometimes do not respond well to taking large doses of inositol mixed in a drink. Large amounts of inositol, e.g., 9,000 mg to 32,400 mg per day, may be difficult for parents to premeasure with precision and to carry in the standard large bottles that contain the inositol powder when the child is not home. Some may opt to carry premeasured doses of inositol in, e.g., sandwich bags or the like, when on the go. However, this may appear suspicious, since the powder has the visual appearance of cocaine. Moreover, children also may refuse to finish the drink or food into which the inositol is mixed or refuse to eat/drink the mixture altogether when the child realizes that he/she is consuming medication. Inositol mixed into liquid also sits at the bottom of the cup, like sand. This type of texture is distasteful to children, particularly those with ASD. The bottom line is that pharmacotherapy is not effective if it cannot be administered and inositol in its typical commercial form can be problematic in this regard, particularly due to the large amounts necessary to achieve therapeutic efficacy.

There is thus a need for an improved method for delivering high doses of inositol to a patient in need thereof. This need extends to any such patients, whether suffering from a DSM 5.0 recognized psychiatric condition (e.g., ASD) or not. For example, whether or not symptomology is rooted in ASD, ADHD, a different condition, or no recognized or diagnosed underlying condition, there is a need for an improved method for delivering high doses of inositol in patients exhibiting one or more of the following symptoms: anxiety, hypersensitivity, restricted areas of interest, repetitive behaviors, insomnia, constipation, hyperfocus, irritability and emotional lability. There is further a need for an improved method and oral dosage form for delivering large quantities of granular or powdered active pharmacological agents in relatively precise amounts.

### BRIEF SUMMARY OF THE INVENTION

Accordingly, in one aspect, the present invention is directed to a method for treating a patient having ASD. As used herein, the term ASD is defined as the diagnostic criteria for DSM 5.0 as published by the American Psychiatric Association. The method includes administering to the patient a therapeutically effective amount of inositol, in one or more, optionally a plurality of comestible units, e.g., baked goods such as cookies. A therapeutically effective amount of inositol is an amount that will reduce one or more core symptoms of ASD or associated ASD symptoms. Thus, in one aspect, the method includes administering to the ASD patient one comestible unit or optionally a plurality of comestible units, e.g., cookies, cumulatively comprising a therapeutically effective amount of inositol. Optionally, the plurality of comestible units comprising inositol may be administered to a patient having ASD in combination with a therapeutically effective amount of an alpha-2 adrenergic agonist in an extended release dosage form.

In another aspect, the present invention is directed to a method for reducing, to a clinically meaningful degree, one or more symptoms associated with ASD in a patient having ASD. The method includes administering to the patient a maximum effective dose of inositol. The maximum effective dose of inositol is determined by providing an amount of inositol to the patient that induces diarrhea and then titrating down to a lower dose that does not induce diarrhea but is immediately below a dose which does induce diarrhea. As used herein in the foregoing context, the term “immediately below” means less than an amount which induces diarrhea, although not substantially less that amount. For example, “immediately below” may mean within 10% of the amount of inositol that induces diarrhea in the patient. The maximum effective dose according to this method is the lower dose.

In another aspect, the present invention is directed to a method for reducing, to a clinically meaningful degree, one or more symptoms associated with ASD in a patient having ASD, e.g., part B of the DSM 5.0 definition of ASD and/or emotional lability/irritability. The method comprises administering to the patient a therapeutically effective amount of inositol, wherein the therapeutically effective amount of inositol is from about 9,000 mg to about 32,400 mg per day. This may be achieved, e.g., through administering one comestible unit or optionally a plurality of comestible units, each comprising a predetermined amount of inositol.

In another aspect, the present invention is directed to a method for treating a patient having one or more of the following symptoms, irrespective of whether such symptoms are rooted in an underlying psychiatric disorder or a neurological disorder: anxiety, hypersensitivity, restricted areas of interest, repetitive behaviors, insomnia, constipation, hyperfocus, irritability and emotional lability. The method includes administering to the patient a therapeutically effective amount of inositol, preferably in one or more comestible units, e.g., baked goods such as cookies. A therapeutically effective amount of inositol, in the context of this aspect of the invention is an amount that will reduce one or more of the aforementioned symptoms. Such therapeutically effective amount of inositol may be from about 9,000 mg to about 32,400 mg per day, optionally from 12,000 mg to 32,400 mg per day. Thus, in one aspect, the method includes administering one or more comestible units, e.g., cookies, cumulatively comprising a therapeutically effective amount of inositol, to a patient having one or more of: anxiety, hypersensitivity, restricted areas of interest, repetitive behaviors, insomnia, constipation, hyperfocus, irritability and emotional lability. In another aspect, the present invention is directed to a method for treating a patient in need of a calming effect. The method includes administering to the patient a therapeutically effective amount of inositol, preferably in a plurality of comestible units, e.g., baked goods such as cookies. Such therapeutically effective amount of inositol may be from about 9,000 mg to about 32,400 mg per day, optionally from 12,000 mg to 32,400 mg per day, optionally from 15,000 mg to 32,400 mg per day, optionally from 18,000 mg to 32,400 mg per day, optionally at least 12,000 mg per day, optionally at least 15,000 mg per day, optionally at least 18,000 mg per day.

In another aspect, the present invention is directed to a method for treating a patient. The method includes administering to the patient a therapeutically effective amount of inositol, the therapeutically effective amount of inositol being at least 12,000 mg per day. The therapeutically effective amount of inositol is cumulatively provided in one or more inositol-containing comestible units per day. Each comestible unit has a predetermined amount of inositol. The one or more inositol-containing comestible units are prepared as a formulation that is subjected to a cooking, boiling or baking process. The method is effective in treating one or more of the following symptoms or disorders in the patient, including: autism spectrum disorder, attention deficit hyperactivity disorder, anxiety, depression, panic disorder, obsessive compulsive disorder, hypersensitivity, restricted areas of interest, repetitive behaviors, irritability and emotional lability.

Optionally, in any embodiment, the selected method provides a therapeutic effect on the patient after two weeks of consistent practicing of the method on the patient.

Optionally, in any embodiment, inositol provides a sweetening effect to the comestible unit, functioning as a sugar substitute in addition to providing a therapeutic effect.

Optionally, in any embodiment, the inositol-containing comestible units are baked goods.

Optionally, in any embodiment, the inositol-containing comestible units are prepared in a cooking or baking process wherein the units are heated in an oven environment that is set at 212° F. or higher for at least five minutes, optionally that is set at 300° F. or higher for at least eight minutes. Optionally, a cooking process involves pan frying. Optionally, in any embodiment, the inositol-containing comestible units are prepared in a boiling process that involves boiling a solution that includes the inositol or mixing a boiling liquid with the inositol.

Optionally, the therapeutically effective amount of inositol is from 18,000 mg to 32,400 mg per day, the plurality of inositol-containing comestible units are cookies, wherein each cookie contains from 2,500 to 4,500 mg of inositol and two to four cookies are administered to the patient two or three times daily.

Optionally, in any embodiment, the inositol in the plurality of inositol-containing comestible units does not have reduced efficacy in treating the patient due to the inositol being exposed to high temperatures during a boiling, cooking or baking process, compared to an identical dose of inositol administered in raw powder form.

Optionally, the inositol-containing comestible units are gummies, candies or lozenges.

Optionally, the therapeutically effective amount of inositol is at least 12,000 mg per day and is provided in a single inositol-containing comestible unit, rather than a plurality. Alternatively, the therapeutically effective amount of inositol is at least 12,000 mg per day, provided in a plurality of inositol-containing comestible units.

Optionally, in any embodiment, at least a portion of the inositol is in an extended release form. Such a form may help to prolong the therapeutic effect and reduce likelihood of diarrhea in the patient.

Optionally, in any embodiment, a therapeutically effective amount of inositol may be from about 9,000 mg to about 32,400 mg per day, optionally from 12,000 mg to 32,400 mg per day, optionally from 15,000 mg to 32,400 mg per day, optionally from 18,000 mg to 32,400 mg per day, optionally at least 12,000 mg per day, optionally at least 15,000 mg per day, optionally at least 18,000 mg per day.

### DETAILED DESCRIPTION OF THE INVENTION

Whether administered as a monotherapy for ASD or in combination with an extended released alpha-2 adrenergic agonist, or for the treatment of other symptoms (which may or may not be rooted in ASD or another underlying psychiatric or neurological disorder), inositol according to the present invention is preferably administered via comestible units, as described below.

As used herein, the phrase “greater efficacy in reducing” with respect to one or more symptoms, means that based on a psychiatrist's qualitative evaluation using ordinary skill and/or evaluation using a recognized quantitative scale in the field of psychiatry, optionally the Social Responsiveness Scale (“SRS,” explained more fully below), the improvement in the one or more symptoms is greater, and preferably unexpectedly greater using the inositol-containing comestible, than a placebo (e.g., placebo comestible).

The phrase “to a clinically meaningful degree” as used herein means noticeable reduction in a given symptom based on a psychiatrist's qualitative evaluation using ordinary skill and/or evaluation using a recognized quantitative scale in the field of psychiatry, optionally the SRS. Other scales for repetitive behavior (e.g., for OCD) are they YBCOS and, for children, the CYBOCS. The method includes reducing, to a clinically meaningful degree, part B of the DSM 5.0 definition of ASD and/or emotional lability/irritability in a patient having ASD. In another aspect, the method includes reducing, to a clinically meaningful degree, one or more of the following symptoms, irrespective of whether such symptoms stem from a DSM-recognized psychiatric disorder or neurological disorder: anxiety, hypersensitivity, restricted areas of interest, repetitive behaviors, insomnia, constipation, hyperfocus, irritability and emotional lability. In another aspect, the method includes providing, to a clinically meaningful degree, a calming effect to a patient in need thereof (whether or not the patient suffers from a DSM-recognized psychiatric disorder or neurological disorder).

Applicant has discovered that high doses of inositol in combination with KAPVAY® or INTUNIV® has helped patients suffering from ADHD more than just KAPVAY® or INTUNIV® alone. This is particularly surprising in light of peer reviewed literature suggesting that inositol actually worsens symptoms in ADHD patients. Some of Applicant's findings in this regard are included in Examples provided below.

In any embodiment, the method comprises administering to a patient one or more inositol-containing comestible units, wherein the one or more inositol-containing comestible units each comprises a predetermined amount of inositol. Optional inositol dosing per comestible unit is described below.

### Inositol Chemistry and Dosing in ASD

Inositol or cyclohexane-1,2,3,4,5,6-hexol is a chemical compound with formula C₆H₁₂O₆or (—CHOH—)₆, a sixfold alcohol (polyol) of cyclohexane. Inositol exists in nine possible stereoisomers. The most prominent form, widely occurring in nature, is cis-1,2,3,5-trans-4,6-cyclohexanehexol, or myo-inositol (former name meso-inositol). Inositol has been shown to have a taste that is half the sweetness of table sugar. For this reason, the Applicant has found the taste of inositol to be well tolerated by ASD patients or other children who are very particular and who thus do not respond favorably to medications with strong tastes or smells.

In addition, Applicant has found that inositol, in high amounts (as disclosed herein) can also serve as a sugar substitute in comestible units, e.g., cookies. In this way, the inositol serves a dual role as both an active agent and a sweetener in a cookie or other comestible unit. This renders the units very effective as a means for drug delivery for children and for simultaneously providing a reduced sugar snack.

The isomer myo-inositol—again the most prominent form of naturally occurring inositol—is a meso compound which has an optically inactive plane of symmetry through the molecule. Besides myo-inositol, the other naturally occurring (albeit uncommon) stereoisomers are scyllo-, muco-, D-chiro-, and neo-inositol. Other isomers are L-chiro-, allo-, epi-, and cis-inositol.

The structure of the most common natural form of inositol, myo-inositol, is shown below:

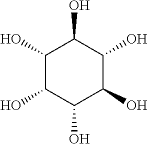 [SMILES: O[C@H]1[C@H](O)[C@@H](O)[C@H](O)[C@@H](O)[C@H]1O]

A preferred form of inositol for use according to some embodiments of the present invention is myo-inositol sold in powder form under the name FREEDA®. Unless otherwise stated in this specification, the term “inositol” or “standard inositol” refers to myo-inositol having a potency substantially similar to that of the inositol powder referenced above, e.g., sold under the name FREEDA®, or other brands and dosage forms having substantially similar potencies. Therapeutically effective doses of inositol (e.g., in powder form) for patients with ASD may generally range from about 4,500 mg to about 32,400 mg per day and preferably from about 9,000 mg to about 32,400 mg per day and especially preferably from about 12,000 mg to about 32,400 mg per day, or optionally from about 12,000 mg to about 24,000 mg per day, or optionally from about 12,000 to about 18,000 mg per day. Optionally, a therapeutically effective dose of inositol is at least 9,000 mg.

Once daily administration of inositol within the foregoing dosage amounts is within the scope of the invention. However, twice daily or three times daily administration is preferred. For example, if a preferred daily dose of inositol for a given patient is 21,000 mg, it is preferred that the inositol be administered to the patient, e.g., 10,500 mg in the morning and 10,500 mg in the evening (twice daily dosing) or 7,000 in the morning, 7,000 around lunch time and 7,000 in the evening (three times daily dosing).

The above recited dosing of inositol may also be effective in treating one or more of the following symptoms, irrespective of whether such symptoms stem from a DSM-recognized psychiatric disorder or neurological disorder: anxiety, hypersensitivity, restricted areas of interest, repetitive behaviors, irritability and emotional lability. The above recited dosing of inositol may also be effective in providing a calming effect to a patient in need thereof. Optionally, in any embodiment, the patient experiences a therapeutic effect after two weeks of consistent inositol administration.

In treating ASD patients with inositol, the Applicant has discovered, among other things, the following noteworthy phenomena:
(1) Inositol may be safely administered, with only the minimal side effect of diarrhea, in doses that significantly exceed doses given to some non-ASD patients described in the literature.
(2) By and large, children (i.e., under age 18) as a population tend to tolerate higher doses of inositol than adults as a population, in terms of the side-effect of diarrhea. For example, as a population, children tend to respond well to about 9,000 mg or more of inositol twice a day, whereas adults tend to tolerate (but at the same time, only require) about half as much. A minority of patients, especially in the adult population, experience diarrhea with much lower doses of inositol. For example, the Applicant has treated patients for whom about 500 mg per day was the maximum dose that would avoid diarrhea, yet that dose was still therapeutically effective in those patients.
(3) The Applicant has found a correlation between the maximum effective dose of inositol in an ASD patient and the point at which the inositol induces diarrhea in the patient. In other words, once a patient experiences diarrhea from inositol, the point of diminishing returns has been reached, as it appears to the Applicant that increasing the inositol dose at that point will not further reduce symptoms to a clinically meaningful degree. While the Applicant would not recommend deliberately inducing diarrhea in a patient for ethical reasons, it is believed that the maximum effective dose of inositol for a given patient is immediately below an amount that induces diarrhea in the patient. The preferred therapeutically effective dose of inositol is the maximum effective dose, although the present invention may include doses of inositol that are therapeutically effective and below the maximum effective dose. While it is not practical to dose titrate patients on inositol by altering the hundreds or more likely thousands of milligram doses of inositol one milligram at a time, one may alter the dose by tens or more likely hundreds of milligrams at a time to determine the maximum effective dose of inositol. For example, in one aspect of the present invention, the maximum effective dose of inositol for a given patient is determined by providing an amount of inositol to the patient that induces diarrhea and then titrating down to a lower dose that does not induce diarrhea but is immediately below a dose which does induce diarrhea, wherein the maximum effective dose is the lower dose. A physician may titrate down, e.g., in increments of 5% or 10% of the amount of inositol that induces diarrhea in the patient. Optionally, the maximum effective dose is within 10% of the amount of inositol that induces diarrhea in the patient. In such an embodiment, for example, the maximum effective dose for a patient who experienced diarrhea at 18,000 mg of inositol per day may be immediately below 18,000 mg, including as low as 16,200 mg.

Even though these findings were based on administration to ASD patients, it is contemplated that the same general dosing guidelines can be used in treating ADHD, OCD, depression, panic disorder, or one or more of the following symptoms, irrespective of whether such symptoms stem from a DSM-recognized psychiatric disorder or neurological disorder: anxiety, hypersensitivity, restricted areas of interest, repetitive behaviors, insomnia, constipation, hyperfocus, irritability and emotional lability. Likewise, it is contemplated that the same general dosing guidelines can be used to provide a calming effect to a patient.

While it is currently preferred that the inositol is administered within the nominal dosage ranges described above, it is contemplated that the potency of inositol may be increased such that it may be administered in lesser nominal amounts than described above with reference to standard inositol, but still provide therapeutically equivalent effectiveness within the scope of the present invention. For example, the Applicant contemplates that inositol could be effectively administered in two to three times lower doses than the nominal amounts described above according to the teachings of U.S. Pat. No. 8,557,792, the entirety of which is incorporated herein by reference. That patent discloses a vitamin B12 formulation and inositol is considered to be part of the vitamin B complex.

Optionally, in any embodiment, at least a portion (optionally, half) of the inositol in the inositol-containing comestible units, is provided in an extended release form. For example, while liquid dosage forms are not preferred vis-à-vis the invention, it is contemplated that aspects of the extended release technology described in U.S. Pat. Nos. 8,062,667 and 8,287,903, which are incorporated by reference herein in their entireties, could be utilized to provide inositol in an extended release form and incorporated into solid or semi-solid comestible units. Other coating technology may also be used to provided inositol in an extended release form according to optional aspects of the invention.

Preferably, in accordance with an aspect of the present invention, the inositol powder is mixed into comestible units, such as cookie dough, in predetermined amounts adapted to provide therapeutic doses of inositol to a patient through administration of a plurality of such units to the patient. These comestible units are discussed in greater detail, below.

### Comestible Units Comprising Inositol

In one aspect, the present invention is directed to a method for reducing, to a clinically meaningful degree, one or more symptoms associated with ASD in a patient having ASD, e.g., part B of the DSM 5.0 definition of ASD and/or emotional lability/irritability. Alternatively, the present invention is directed to treating ADHD, OCD, Depression or Panic Disorder. Alternatively, the present invention is directed to treating one or more of the following symptoms, irrespective of whether such symptoms stem from a DSM-recognized psychiatric disorder or neurological disorder: anxiety, hypersensitivity, restricted areas of interest, repetitive behaviors, insomnia, constipation, hyperfocus, irritability and emotional lability. In any case, the method comprises administering to the patient a therapeutically effective amount of inositol, wherein the therapeutically effective amount of inositol is from about 9,000 mg to about 32,400 mg per day, preferably 12,000 mg to 32,400 mg per day. This may be achieved, e.g., through administering one or more comestible units cumulatively comprising a predetermined therapeutic amount of inositol to a patient.

The comestible unit is a solid or substantially solid food, as opposed to a drink. The comestible unit is preferably a baked good, candy or other food that is appealing to ASD or other patients, especially children, who are particularly sensitive to bad tastes and odors. Optionally, the comestible unit is a comestible suspension which is solid or semi-solid at room temperature, optionally wherein the inositol is suspended in the unit homogenously. Optionally, such suspension is processed in some sort of heating process (e.g. boiling process), as discussed herein. Preferably, the comestible unit is a cookie or other small measured quantity of food that is not overly filling per unit. In this way, a patient may consume therapeutic doses of inositol by consuming a plurality of such units. Preferably, a comestible unit is packaged together with a plurality of other similarly sized units, wherein every unit contains substantially the same amount of inositol. In this way, a patient or caretaker does not need to premeasure inositol powder for each administration and mix the powder into a delivery liquid (e.g., juice) or semi-solid food (e.g., applesauce). The comestible units thus simplify administration of inositol and make taking the medicine an enjoyable experience for the patient. As an alternative, a single comestible unit may itself provide all of the predetermined therapeutic amount of inositol (e.g., at least 12,000 mg).

Optionally, each comestible unit (e.g., cookie) includes predetermined amounts of from 1,000 to 7,000 mg of inositol, optionally from 1,500 to 6,000 mg of inositol, optionally from 2,000 to 5,500 mg of inositol, optionally from 2,000 to 4,500 mg of inositol, optionally from 2,000 to 3,000 mg of inositol, optionally from 2,500 to 4,500 mg of inositol, optionally from 2,500 to 3,500 mg of inositol, optionally from 3,000 to 4,500 mg of inositol, or optionally precisely or about (i.e., plus or minus 50 mg) any of the following amounts of inositol: 1,500 mg, 1,600 mg, 1,700 mg, 1,800 mg, 1,900 mg, 2,000 mg, 2,100 mg, 2,200 mg, 2,300 mg, 2,400 mg, 2,500 mg, 2,600 mg, 2,700 mg, 2,800 mg, 2,900 mg, 3,000 mg, 3,100 mg, 3,200 mg, 3,300 mg, 3,400 mg, 3,500 mg, 3,600 mg, 3,700 mg, 3,800 mg, 3,900 mg, 4,000 mg, 4,100 mg, 4,200 mg, 4,300 mg, 4,400 mg, 4,500 mg, 4,600 mg, 4,700 mg, 4,800 mg, 4,900 mg, 5,000 mg, 5,100 mg, 5,200 mg, 5,300 mg, 5,400 mg, or 5,500 mg.

Since inositol has a sweet taste, the foregoing exemplary amounts of inositol may be used to reduce or replace sugar in comestible units according to the present invention. For example, in one embodiment, the invention may be a cookie made from the following ingredients: unbleached flour, vegetable oil, a predetermined amount of inositol, (optionally) sugar, cocoa, eggs, water, honey, salt, leavening and vanilla, wherein the predetermined amount of inositol per cookie is optionally precisely or about (i.e., plus or minus 50 mg) any of the following: 1,500 mg, 1,600 mg, 1,700 mg, 1,800 mg, 1,900 mg, 2,000 mg, 2,100 mg, 2,200 mg, 2,300 mg, 2,400 mg, 2,500 mg, 2,600 mg, 2,700 mg, 2,800 mg, 2,900 mg, 3,000 mg, 3,100 mg, 3,200 mg, 3,300 mg, 3,400 mg, 3,500 mg, 3,600 mg, 3,700 mg, 3,800 mg, 3,900 mg, 4,000 mg, 4,100 mg, 4,200 mg, 4,300 mg, 4,400 mg, 4,500 mg, 4,600 mg, 4,700 mg, 4,800 mg, 4,900 mg, 5,000 mg, 5,100 mg, 5,200 mg, 5,300 mg, 5,400 mg, or 5,500 mg.

Preferably the comestible units are packaged with a plurality of other such units, with printed indicia on or in a package containing such units, communicating that the units and/or high doses of inositol may be effective in treating ASD or have been shown to be effective in treating ASD and/or one or more of the following symptoms: anxiety, hypersensitivity, restricted areas of interest, repetitive behaviors, insomnia, constipation, hyperfocus, irritability and emotional lability (irrespective of whether such symptoms stem from a DSM-recognized psychiatric disorder or neurological disorder) and/or for providing a calming effect (irrespective of specific symptomology or underlying condition). The package may also provide printed indicia stating that the comestibles, when consumed according to dosing described herein, may provide a therapeutic effect (e.g., the foregoing effects or treatment of symptoms) after two weeks of consistent administration to the patient. For example, a package of inositol-containing cookies may include indicia to any of the aforementioned effects. A package of inositol-containing comestible units may further include printed indicia communicating that the units and/or high doses of inositol may cause diarrhea and/or that the preferred amount of inositol for a given patient is to be provided in one unit less than the number of units daily that induce diarrhea in a given patient.

Aside from cookies, by way of example only, the inositol-containing comestible units may be brownies, pastries, doughnuts, graham crackers, candies, cakes, chocolates, cereal bars, pies, wafers, muffins, cupcakes, gummies and lollipops, comestible suspensions that are solid or semi-solid at room temperature, or other sweet food units.

Optionally, in any embodiment, the inositol-containing comestible units are prepared in a cooking or baking process wherein the units are heated in an oven environment that is set at least 300° F. or higher for at least eight minutes. These temperature and time parameters are merely exemplary of an optional cooking or baking process and it is contemplated that alternative temperatures and/or times may be used. The parameters and conditions may vary depending on the specific type of comestible, the volume of each comestible, etc. For example, an oven environment must typically be set at least at 212° F. to effectively cook or bake and is preferably set at least at 250° F., more preferably at least 275° F., even more preferably at least 300° F. One recipe, for cupcakes, for example, calls for heating cake batter in an oven environment set at 350° F. for about fifteen minutes. Alternatively, to produce hard candy (e.g., lollipops), in a boiling process, a sugar (and/or corn syrup) and water solution must typically be heated to at least 300° F., poured into a mold and then cooled to form the candy. In another embodiment, a semi solid comestible may be made in a boiling process, which may involve mixing boiling or otherwise very hot water with a powdered mixture, e.g., to make a comestible suspension. Any of these processes may be used, according to optional aspects of the invention, to produce inositol-containing comestibles. It has been surprisingly found that subjecting inositol-containing comestibles to high temperatures, e.g., through baking, does not noticeably reduce the therapeutic efficacy of the inositol in the comestibles. This is a surprising finding in view of literature and product warnings associated with inositol, as explained further below.

Optionally, the therapeutically effective amount of inositol is from 18,000 mg to 32,400 mg per day, the plurality of inositol-containing comestible units are cookies, wherein each cookie contains from 2,500 to 4,500 mg of inositol, the method comprising administering to the patient two to four cookies two or three times daily. Preferably, in this and other embodiments disclosed herein, the inositol in the plurality of inositol-containing comestible units does not have reduced efficacy in treating the patient due to the inositol being exposed to high temperatures during baking, compared to an identical dose of inositol administered in raw powder form.

Optionally, in any embodiment, the inositol-containing comestible units are lozenges, gummies or candies.

### Combination Therapy

As discussed above, combination therapy is the subject of Applicant's earlier filed patent applications, including U.S. Pat. Pub. 2014/0309271 (the “'271 Application”). In one embodiment, the inositol-containing comestible units of the present invention may be administered in combination with an alpha-2 adrenergic agonist in an extended release dosage form, to treat ASD, according to methods disclosed in the '271 Application.

### Measurement of Symptoms

It is within the ability of a psychiatrist of ordinary skill to assess each of the various symptoms and diagnostic criteria for ASD generally and for other symptoms associated with ASD (or symptoms unrelated to ASD) described herein, in a clinical setting, using his or her training and experience. Optionally, a psychiatrist may desire to measure these symptoms and criteria using a recognized quantitative scale in the field of ASD or psychiatry generally, optionally the Social Responsiveness Scale (“SRS”). The Social Responsiveness Scale (SRS) (Constantino, J. et al., J Dev Behav Pediatr, 21:2-11 (2000); Constantino, J. et al., J Autism Dev Disord., 3:427-433 (2003)) is a norm-referenced, 65-item report questionnaire developed to measure social behaviors, including social awareness, social information processing, reciprocal social communication, and social anxiety, in both clinical and non-clinical populations. It is designed for use with children ages 4 through 18. The SRS items measure symptoms in the domains of social awareness, social information processing, reciprocal social communication, social anxiety/avoidance, and stereotypic behavior/restricted interests. Each item is scored from 1 (not true) to 4 (almost always true). Scores are obtained for five treatment subscales: Social Awareness (e.g., “Is aware of what others are thinking or feeling”), Social Cognition (e.g., “Doesn't recognize when others are trying to take advantage of him or her”), Social Communication (e.g., “Avoids eye contact or has unusual eye contact”), Social Motivation (e.g., “Would rather be alone than with others”), and Autistic Mannerisms (e.g., “Has an unusually narrow range of interests”).

### Examples of Treatment for Patients

Aspects of the present invention are further explained by the following examples which should not be construed by way of limiting the scope of the present invention.

### Example 1

Cookies are made to contain 2,700 mg of inositol power each (¾ tsp of FREEDA® brand inositol powder having a concentration of 900 mg per ¼ tsp). The inositol acts as a sweetener which allows reduction in sugar content of each cookie.

It is found that preferred dosing of these 2,700 mg inositol cookies is two to four cookies two or three times daily for ASD patients, titrated until a given ASD patient reaches his/her diarrhea threshold. It is found that these inositol-containing cookies at these doses are effective in treating symptoms of ASD, including part B of the DSM 5.0 definition of ASD and emotional lability/irritability.

Optionally, the cookies are taken in combination with therapeutically effective amounts of extended release clonidine (KAPVAY®) or extended release guanfacine (INTUNIV®).

### Example 2

Cookies are made to contain 3,600 mg of inositol power each (1 tsp of FREEDA® brand inositol powder having a concentration of 900 mg per ¼ tsp). The inositol acts as a sweetener which allows reduction in sugar content of each cookie.

It is found that preferred dosing of these 3,600 mg inositol cookies is two to three cookies two times daily for ASD patients, titrated until a given ASD patient reaches his/her diarrhea threshold. It is found that these inositol-containing cookies at these doses are effective in treating symptoms of ASD, including part B of the DSM 5.0 definition of ASD and emotional lability/irritability.

Optionally, the cookies are taken in combination with therapeutically effective amounts of extended release clonidine (KAPVAY®) or extended release guanfacine (INTUNIV®).

### Example 3

In a study, a group of 24 ASD patients are given the cookies of Example 2 above, according to the dosing guidelines described therein (i.e., titrated to each patient's diarrhea threshold). After three weeks, it is observed that 22 out of the 24 patients' ASD symptoms are alleviated, particularly in the domains of part B of the DSM 5.0 definition of ASD and emotional lability/irritability.

The 24 patients are then separated into two groups. Twelve of these patients continue on the same treatment as before. The other twelve are, without their knowledge, switched to cookies that appear and taste essentially identical to the inositol-containing cookies, except that these new cookies (the placebo cookies) contain no inositol. After two weeks, the treated group continues to respond as well as it had after the first three weeks of the study. However, the patients in the placebo group revert back to their pre-treatment symptomology, and in some cases worse than their pre-treatment symptomology.

This example shows how, in one aspect, the invention may provide an effective tool in a clinical trial to render a switch from inositol-containing cookies to placebo cookies imperceptible (in taste, appearance, texture, etc.) to a patient.

### Example 4

Applicant conducted a retrospective analysis of medical charts of his ASD patients who, in addition to having the core symptoms of ASD, also had symptoms associated with ASD (impulsivity, concentration deficit or attention deficit and emotional lability/irritability). 175 of these patients had initially received some form of medication (not treatment according to the present invention) or had been given psychotherapy without medication. Of these, 22% responded in some way to the medication and 17% responded in some way to psychotherapy alone. 77% of those on one or more of the following medications experienced adverse effects: stimulants, SSRI's, SNRI's, mood stabilizers, antipsychotics, benzodiazepines and immediate release alpha-2 agonists.

The retrospective analysis considered four different ASD patient groups, which had been administered one or both agents (i.e., inositol and/or an extended release alpha-2 adrenergic agonist). Group 1 comprised patients who were given inositol alone. Group 2 comprised patients who were given extended release guanfacine HCl alone. Group 3 comprised patients who were given extended release clonidine HCl alone. Group 4 comprised patients who were given inositol in combination with extended release guanfacine HCl or extended release clonidine HCl.

Dosing of the patients varied depending on therapeutic response and side effects (e.g., diarrhea from the inositol). Patients who received inositol generally received anywhere from as little as 7,200 mg to as much as 32,400 mg. Patients who received extended release clonidine HCl generally received anywhere from 0.2 mg to 0.4 mg daily. Patients who received extended release guanfacine HCl generally received anywhere from 1 mg to 4 mg daily.

In this retrospective analysis, based on observations recorded in the Applicant's charts, the he rated the patients' responses to treatment. During the time of evaluation and treatment of the patients whose charts were later reviewed in this retrospective analysis, Applicant had used standard modes of psychiatric evaluation in assessing their condition both pre and post treatment. This included assessment of each patient's familial history, information about the patient's mother's pregnancy, interviews with the patient and his/her parent(s) regarding the patient's behavior, and follow-up assessment of the patient's situation in school and social interactions after receiving treatment.

The following chart summarizes the results collected from this analysis:

| | | Patients Experiencing |
|---|---|---|
| Group | Core Symptom | Marked Improvement |
| 1 (inositol alone) | Particularism and repetitive | 62 of 66 |
| | behaviors | (94%) |
| | (part B of DSM 5.0 ASD) | |
| | Social reciprocity deficits | None |
| | (part A of DSM 5.0 ASD) | |
| 2 (extended release | Particularism and repetitive | None |
| guanfacine HCl | behaviors | |
| alone) | (part B of DSM 5.0 ASD) | |
| | Social reciprocity deficits | 65 out of 88 |
| | (part A of DSM 5.0 ASD) | (74%) |
| 3 (extended release | Particularism and repetitive | None |
| clonidine HCl | behaviors | |
| alone) | (part B of DSM 5.0 ASD) | |
| | Social reciprocity deficits | 17 out of 25 |
| | (part A of DSM 5.0 ASD) | (68%) |
| 4 (extended release | Particularism and repetitive | 33 of 34 |
| guanfacine HCl or | behaviors | (97%) |
| clonidine HCl, in | (part B of DSM 5.0 ASD) | |
| combination with | Social reciprocity deficits | 33 of 34 |
| inositol) | (part A of DSM 5.0 ASD) | (97%) |

The foregoing chart shows that 62 of the 66 patients (94%) receiving inositol as a monotherapy showed improvement in part B of the DSM 5.0 definition of ASD and the associated ASD symptom of emotional lability/irritability. It is also of note that marked improvement of associated symptoms was also observed in 97% of Group 4 patients. Both ASD core and associated symptoms were treated in Group 4 patients without increased incidence of adverse effects found in any other group.

The foregoing chart further shows that the combination of drugs produced a synergistic effect specifically on the core ASD symptom of social reciprocity deficits (part A of DSM 5.0 ASD). Applicant observed that inositol had basically no effect in treating this core symptom. Extended release guanfacine or extended release clonidine as a monotherapy (Groups 2 and 3, respectively) markedly improved that same core symptom in only 74% and 68% of patients, respectively. Yet, 97% of patients administered the combination therapy (Group 4) demonstrated marked improvement in the core symptom of social reciprocity deficits. This means that inositol, which showed basically no effect on social reciprocity deficits when used as a monotherapy, significantly increased the percentage of patients who experienced marked improvement in that core symptom when used in combination with extended release guanfacine or clonidine, compared to patients who used guanfacine or clonidine alone.

While combination therapy was certainly shown to be superior, monotherapy using high doses of inositol was still shown to be effective in treating some symptoms in ASD, as the cart above demonstrates. Further, although inositol in premade (cooked) comestible form was likely not used in any of the patients in this Example, based on other Examples discussed herein and other of Applicant's experiences with baked comestibles, Applicant contemplates that results would have been no different had the inositol been provided in inositol-containing comestible units according to the invention.

### Example 5

A study is conducted involving 28 patients who qualify for an ASD diagnosis. In a first phase, these patients are divided into two groups: a drug group (16 patients) that receives therapeutic amounts of inositol (average from 9,000 mg to 18,000 mg per day) in an oral powder dosage form for two weeks; and a placebo group (12 patients) that receives a confectionary sugar-based powdered placebo (intended to represent inositol) for two weeks. Neither group receives any other psychiatric drug during the two weeks of drug or placebo administration. After those two weeks, the progress of the patients in the two groups are measured according to the SRS. It is found that all 16 patients in the drug (inositol alone) group show clinically meaningful reduction in the restricted areas of interest and repetitive behaviors subscale of the SRS. However, none of the 12 patients in the placebo group show any measurable reduction in the restricted areas of interest and repetitive behaviors subscale of the SRS.

### Example 6

The Applicant's preferred brand of inositol, FREEDA®, provides information on the bottle stating that the serving size for adults is 900 mg, that the bottle should be kept out of the reach of children, that the product should be stored at room temperature and that the product should not be exposed to excessive heat or moisture. Notwithstanding these statements, Applicant determined that approximately twenty times the specified serving size for adults is safe and effective for treating children having ASD or otherwise experiencing anxiety, hypersensitivity, restricted areas of interest, repetitive behaviors, irritability and emotional lability (irrespective of whether such symptoms are rooted in an underlying psychiatric disorder or neurological disorder). Applicant further found that these large doses of inositol surprisingly retained their efficacy when mixed into hot liquids or semi-solids (e.g., oatmeal).

Based on these findings and in view of difficulties associated with administering large amounts of inositol powder to children, Applicant worked with a cookie company to try to fit as much inositol in standard sized cookies as possible. Applicant set out to make the cookie retain a good taste and texture so as to render the presence of the inositol virtually unnoticeable to a child eating the cookie. Applicant and the company ultimately created a cookie (sold under the name CALMINTOL) that included 3,165 mg inositol per cookie. The CALMINTOL package states that there are three cookies per serving (i.e., 9,495 mg per serving). Applicant recommends two servings per day for patients in need thereof (e.g., approximately 20,000 mg daily, give or take).

It was found that these cookies treated ASD symptoms as well as other symptoms that may or may not be rooted in ASD or another underlying psychiatric or neurological condition. It is contemplated that these cookies are useful in treating a patient having one or more of the following symptoms: anxiety, hypersensitivity, restricted areas of interest, repetitive behaviors, irritability and emotional lability, or otherwise providing a calming effect to a patient in need thereof (irrespective of whether such symptoms stem from a DSM-recognized psychiatric disorder or neurological disorder).

The cookie company was initially skeptical about making this cookie because it is extremely unconventional in the food industry to incorporate such high pharmacological doses of active agents in foods, especially foods primarily intended for children. This skepticism is based on several factors. For one, while food may often be “fortified with” or naturally contain low levels of vitamins or minerals, food is not typically used as a delivery vehicle for pharmacological doses of active substances. For example, inositol is provided in infant formula in amounts less than 5 mg. per 5 fl. oz. serving. Moreover, the FREEDA® inositol bottle provides that the serving size for adults is 0.9 grams and the bottle prominently says, “Keep out of the reach of children.” In addition, the bottle warns that the product should be stored at room temperature and should not be “expose[d] to excessive heat or moisture.” Based on these factors, a skilled artisan would reasonably expect that the inositol dosing of the cookies is excessive for children and that the inositol's potency would be adversely affected by the extreme conditions to which it would be subject during the baking process. Nevertheless, it has been surprisingly found that the inositol in fact did not measurably lose efficacy in the baking process and that the CALMINTOL cookies successfully and safely treated symptoms in pediatric patients.

### Example 7

A 10 year old male patient meets DSM 5.0 criteria for Attention Deficit Hyperactivity Disorder-combined type with associated symptoms of ego-syntonic hyperfocus on restricted areas of interest (computer games). The patient exhibits hypersensitivity to touch, smell, and taste without Obsessive Compulsive Disorder and irritability/emotional lability without symptoms of Major Depressive Disorder, Bipolar Disorder, Oppositional Defiant Disorder, Conduct Disorder, or Disruptive Mood Disregulation Disorder. Social reciprocity is maintained, thus ruling out Social Communication Disorder or ASD.

The patient fails first-line ADHD psychostimulant treatments including Ritalin, Ritalin LA, Focalin, Focalin XR, Concerta, Quillivant, Daytrana, Dexedrine tablet and capsule, Adderall, Adderall XR, and Vyvanse. These drugs exacerbate hyperfocus on restricted areas of interest, exacerbate irritability/emotional lability, suppress an already poor appetite which diminishes weight and growth progression, and produce new onset insomnia with associated fatigue and further irritability. Tenex and Clonidine produce sedation and hypotension. Clonidine patch is discontinued due to skin hypersensitivity. Straterra, Wellbutrin XL, and Nuvigil produce no effect.

The patient's ADHD symptoms respond well to INTUNIV® 2 mg at bedtime which improved all core symptoms without exacerbating associated symptoms of hyperfocus, hypersensitivity and irritability/emotional lability. The patient does not tolerate higher doses of INTUNIV® due to sedation and lethargy.

The patient fails trials of SSRI's, including Prozac, Zoloft, and Lexapro, in an attempt to reduce hyperfocus, due to activation, insomnia and worsening irritability/emotional lability. Risperdal and Abilify are poorly tolerated due to sedation, gynecomastia on Risperdal, and failure to improve core ADHD symptoms

The addition of inositol powder is administered via 3 inositol-containing cookies twice a day according to the disclosed concept, for a total of 19,000 mg daily. This alleviates ADHD associated symptoms of hyperfocus, hypersensitivity, restricted areas of interest, and irritability/emotional lability. Inositol has no negative side effects, neither on reducing appetite nor causing weight loss or gain. Appetite is improved due to a reduction in hypersensitivity with respect to food tastes and textures. Social interactions are also improved due to an overall calming effect of the INTUNIV® and inositol combination. The patient continues to do well for 3 years on the combination of INTUNIV® for core symptoms of ADHD and inositol for associated symptoms of ADHD without new side effects or need for dose escalation.

### Example 8

A 13 year old female patient has DSM 5.0 Attention Deficit Hyperactivity Disorder-inattentive type with associated symptoms of restricted areas of interest-ego syntonic perfectionism in her school studies, irritability/emotional lability, insomnia with fatigue, and constipation. She does not meet criteria for other psychiatric diagnoses. The patient is tried on but fails psychostimulants due to anxiety, new onset eye blinking tics, worsening constipation and insomnia with fatigue. The patient is tried on but discontinues S SRI's due to behavioral disinfection, activation and aggression. The patient is tried on Risperdal and Abilify but discontinues those drugs due to exacerbated concentration and excessive weight gain refractory to diet and exercise.

The patient's ADHD symptoms respond well to INTUNIV® 4 mg in the morning. The addition of the same regimen of inositol cookies (as set forth in Example 7, above) alleviates constipation, insomnia, restricted areas of interest. The inclusion of inositol in the patient's therapy improves her social functioning by reducing her overall stubbornness and rigidity, and by enabling flexibility and fluidity required for social interactions. After a period of time on treatment, the patient continues to do well on the combination with no side effects or need for dose escalation.

### Example 9

This example demonstrates how inositol powder addresses side effects seen in ADHD patients treated with extended release alpha-2 adrenergic agonists. A 7 year old female patient is diagnosed with ADHD-combined type. She is successfully treated for both core symptoms of ADHD with INTUNIV® 1 mg every morning. However, the patient develops new side effects, ostensibly from the INTUNIV®, including: constipation, insomnia, hyperfocus on restricted areas of interest (such as on babies) and irritability/emotional lability. The INTUNIV® improves the patient's social functioning by enabling her to focus better in conversations. However, the hyperfocus on her personal idiosyncratic interests, apparently due to the INTUNIV® simultaneously reduces her empathy for others. Adding inositol-containing cookies as described above in Examples 7 and 8 alleviate the following side effects: constipation, insomnia, restricted areas of interest, and irritability/emotional lability. The inositol also improves her social reciprocity by enabling her to be less rigidly preoccupied with her own interests.

While the invention has been described in detail and with reference to specific examples thereof, it will be apparent to one skilled in the art that various changes and modifications can be made therein without departing from the spirit and scope thereof.

What is claimed is:

1. A method for treating a patient, the method comprising: administering to the patient a therapeutically effective amount of inositol, the therapeutically effective amount of inositol being at least 12,000 mg per day, the therapeutically effective amount of inositol being cumulatively provided in one or more inositol-containing comestible units per day, each comestible unit comprising a predetermined amount of inositol, wherein the one or more inositol-containing comestible units are prepared as a formulation that is subjected to a cooking, boiling or baking process, the method being effective in treating one or more symptoms or disorders selected from the group consisting of: autism spectrum disorder, attention deficit hyperactivity disorder, anxiety, depression, panic disorder, obsessive compulsive disorder, hypersensitivity, restricted areas of interest, repetitive behaviors, insomnia, constipation, hyperfocus, irritability and emotional lability.

2. The method of claim 1, wherein the predetermined amount of inositol is selected from an amount consisting of about: 1,500 mg, 1,600 mg, 1,700 mg, 1,800 mg, 1,900 mg, 2,000 mg, 2,100 mg, 2,200 mg, 2,300 mg, 2,400 mg, 2,500 mg, 2,600 mg, 2,700 mg, 2,800 mg, 2,900 mg, 3,000 mg, 3,100 mg, 3,200 mg, 3,300 mg, 3,400 mg, 3,500 mg, 3,600 mg, 3,700 mg, 3,800 mg, 3,900 mg, 4,000 mg, 4,100 mg, 4,200 mg, 4,300 mg, 4,400 mg, 4,500 mg, 4,600 mg, 4,700 mg, 4,800 mg, 4,900 mg, 5,000 mg, 5,100 mg, 5,200 mg, 5,300 mg, 5,400 mg, and 5,500.

3. The method of claim 1, wherein the predetermined amount of inositol is from 3,000 to 6,000 mg of inositol.

4. The method of claim 1, wherein the plurality of inositol-containing comestible units are selected from the group consisting of: cookies, brownies, pastries, doughnuts, graham crackers, cakes, chocolates, cereal bars, pies, wafers, muffins and cupcakes.

5. The method of claim 1, wherein the plurality of inositol-containing comestible units are cookies, wherein each cookie contains from 2,500 to 4,500 mg of inositol, the method comprising administering to the patient two to four cookies two or three times daily.

6. The method of claim 4, comprising administering to the patient two to four comestible units two or three times daily.

7. The method of claim 6, wherein each comestible unit contains from 2,500 to 4,500 mg of inositol.

8. The method of claim 1, wherein the therapeutically effective amount of inositol is up to 32,400 mg per day.

9. The method of claim 1, wherein the therapeutically effective amount of inositol is from 12,000 mg to 32,400 mg per day.

10. The method of claim 1, wherein the therapeutically effective amount of inositol is from 18,000 mg to 32,400 mg per day.

11. The method of claim 1, wherein the therapeutically effective amount of inositol is from 18,000 mg to 32,400 mg per day, the plurality of inositol-containing comestible units are cookies and each cookie contains from 2,500 to 4,500 mg of inositol, the method comprising administering to the patient two to four cookies two or three times daily.

12. The method of claim 12, wherein the method induces diarrhea in the patient and wherein the diarrhea is alleviated by reducing the amount of inositol administered to a maximum effective dose of inositol, the method providing a therapeutic effect which is not diminished at the maximum effective dose compared to a therapeutic effect the patient experienced when the method induced diarrhea in the patient.

13. The method of claim 1, wherein the inositol-containing comestible units are baked goods, wherein the inositol in the plurality of inositol-containing comestible units does not have reduced efficacy in treating the patient due to the inositol being exposed to high temperatures during baking, compared to an identical dose of inositol administered in raw powder form.

14. The method of claim 1, wherein the inositol-containing comestible units are baked goods.

15. The method of claim 1, wherein the inositol-containing comestible units are prepared in a cooking or baking process wherein the units are heated in an oven environment that is set at least 300° F. or higher for at least eight minutes.

16. The method of claim 15, wherein the therapeutically effective amount of inositol is from 18,000 mg to 32,400 mg per day, the plurality of inositol-containing comestible units are cookies, wherein each cookie contains from 2,500 to 4,500 mg of inositol, the method comprising administering to the patient two to four cookies two or three times daily.

17. The method of claim 15, wherein the inositol in the plurality of inositol-containing comestible units does not have reduced efficacy in treating the patient due to the inositol being exposed to high temperatures during the cooking or baking process, compared to an identical dose of inositol administered in raw powder form.

18. The method of claim 1, wherein the inositol-containing comestible units are gummies or candies.

19. The method of claim 1, wherein the therapeutically effective amount of inositol is provided in a single comestible unit per day.

20. The method of claim 1, wherein at least a portion of the therapeutically effective amount of inositol is provided in an extended release form.

21. A method for treating a patient in need of a calming effect, the method comprising: administering to the patient a therapeutically effective amount of inositol, the therapeutically effective amount of inositol being at least 12,000 mg per day, the therapeutically effective amount of inositol being cumulatively provided in one or more inositol-containing comestible units per day, each comestible unit comprising a predetermined amount of inositol, wherein the one or more inositol-containing comestible units are prepared as a formulation that is subjected to a cooking, boiling or baking process, the method being effective in providing a calming effect to a patient in need thereof.

22. The method of claim 21, wherein the plurality of inositol-containing comestible units are cookies, wherein each cookie contains from 2,500 to 4,500 mg of inositol, the method comprising administering to the patient two to four cookies two or three times daily.

23. The method of claim 21, wherein the therapeutically effective amount of inositol is up to 32,400 mg per day.

24. The method of claim 21, wherein the therapeutically effective amount of inositol is from 18,000 mg to 32,400 mg per day.

25. The method of claim 21, wherein the therapeutically effective amount of inositol is from 18,000 mg to 32,400 mg per day, the plurality of inositol-containing comestible units are cookies and each cookie contains from 2,500 to 4,500 mg of inositol, the method comprising administering to the patient two to four cookies two or three times daily.

26. The method of claim 21, wherein the inositol-containing comestible units are baked goods, wherein the inositol in the plurality of inositol-containing comestible units does not have reduced efficacy in treating the patient due to the inositol being exposed to high temperatures during baking, compared to an identical dose of inositol administered in raw powder form.

27. The method of claim 21, wherein the inositol-containing comestible units are prepared in a cooking or baking process wherein the units are heated in an oven environment that is set at least 300° F. or higher for at least eight minutes.
